(19) United States
(12) Patent Application Publication
(10) Pub. No.: US 2024/0423245 A1
(43) Pub. Date: Dec. 26, 2024

(54) CANNABINOIDS FOR IMPROVEMENT OF PERFORMANCE IN LIVESTOCK

(71) Applicant: Kansas State University Research Foundation, Manhattan, KS (US)

(72) Inventors: Michael D. Kleinhenz, Manhattan, KS (US); Johann F. Coetzee, Manhattan, KS (US); Jason Griffin, Haysville, KS (US)

(21) Appl. No.: 18/694,007

(22) PCT Filed: Sep. 21, 2022
(86) PCT No.: PCT/US2022/044195

§ 371 Date: Mar. 21, 2024

Related U.S. Application Data
(60) Provisional application No. 63/246,370, filed on Sep. 21, 2021.

(51) Int. Cl. A23K 20/111; A23K 50/10; A61K 31/00

(52) U.S. Cl. CPC A23K 20/111; A23K 50/10; A61K 31/658

(57) ABSTRACT

Methods of improving performance, minimizing negative effects of stressful stimuli, or reducing stress response, improving behavior, immune function, morbidity, performance, and/or carcass characteristics in livestock. The methods comprise administering an effective amount of cannabinoids to an animal before, during and/or after exposure to a stressful event, wherein said cannabinoids are administered to the animal for a total time period of up to 14 days. The present disclosure also concerns animal feeds or feed additives for use in the methods, wherein the animal feed or feed additive comprises an effective amount of cannabinoids.

### CROSS-REFERENCE TO RELATED APPLICATIONS

The present application claims the priority benefit of U.S. Provisional Patent Application Ser. No. 63/246,370, filed Sep. 21, 2021, entitled CANNABINOIDS FOR IMPROVEMENT OF PERFORMANCE OF LIVESTOCK, incorporated by reference in its entirety herein.

### FEDERALLY SPONSORED RESEARCH OR DEVELOPMENT

This invention was made with government support under AFRI No. 2020-67030-31479, awarded by the United States Department of Agriculture and the National Institute of Food and Agriculture. The government has certain rights in the invention.

### BACKGROUND AND INTRODUCTION

Handling, transportation, and processing are painful and stressful for livestock. Societal concern about the moral and ethical treatment of animals, including livestock, is becoming more prevalent. Transportation stress and other stressors can result in significant economic losses to producers due to decreased animal productivity and increased medication costs associated with sickness such as bovine respiratory disease (BRD). Most of the approaches to respiratory disease management in cattle are limited to vaccination and antibiotics to decrease disease prevalence and severity. Cattle also experience a characteristic decrease in body weight after transportation known as “shrink.” Transportation of cattle is a necessary part of the production cycle that occurs during significant bovine life events including sorting, weaning, processing, and slaughter. Most cattle are transported by truck at least once during their lifetime. Transit causes homeostatic imbalances that predispose cattle to developing bovine respiratory disease. Prolonged exposure to other stressful stimuli can also result in immune system inhibition. There remains a need to minimize the negative effects of transportation and other stressors on livestock health homeostasis, immune function, and performance.

### SUMMARY

Described herein are methods of improving performance of livestock. The methods generally comprise administering an effective amount of cannabinoids to an animal before, during and/or after exposure to a stressful event. Preferably, the cannabinoids are administered to the animal for a time period of up to 14 days, preferably from 5-14 days.

Also described herein are methods of minimizing negative effects of stressful stimuli in livestock. The methods generally comprise administering an effective amount of cannabinoids to an animal before, during and/or after exposure to a stressful event. Preferably, the cannabinoids are administered to the animal for a time period of up to 14 days, preferably from 5-14 days.

The disclosure also concerns methods for reducing stress response, improving behavior, immune function, morbidity, performance, and/or carcass characteristics in livestock The methods generally comprise administering an effective amount of cannabinoids to an animal before, during and/or after exposure to a stressful event. Preferably, the cannabinoids are administered to the animal for a time period of up to 14 days, preferably from 5-14 days.

Also contemplated herein are animal feeds or feed additives for use in any of the methods disclosed herein. The animal feeds or feed additives are suitable for administration to animals, and generally comprise an effective amount of cannabinoids.

### BRIEF DESCRIPTION OF THE DRAWING FIGURES

FIG. 1 is a graph showing mean plasma concentrations (ng/mL) of cannabidiolic acid (CBDA) at 5.5 mg/kg, cannabidivarinic acid (CBDVA) at 0.02 mg/kg, cannabigerolic acid (CBGA) at 0.2 mg/kg, and tetrahydrocannabinolic acid-A (THCA-A) at 0.1 mg/kg, over time in cattle feed industrial hemp once a day for 14 days (n=8).

FIG. 2 is a graph showing mean lying bouts for cattle fed 25 g industrial hemp (HEMP; n=8) for 14 days compared to cattle not fed industrial hemp (CNTL; n=8).

FIG. 3 is a graph showing overall mean body weight change for steers (n=12) receiving industrial hemp (HEMP) for a target dose of 5.5 mg/kg CBDA or placebo (PLBO) prior to transportation (TRANS) or remaining on premise (STAY). All steers were enrolled in each treatment.

FIG. 4 is a graph showing mean overall cortisol by time for steers for steers (n=12) receiving industrial hemp (HEMP) for a target dose of 5.5 mg/kg CBDA or placebo (PLBO) prior to transportation (TRANS) or remaining on premise (STAY). All steers were enrolled in each treatment.

### DETAILED DESCRIPTION

Described herein are new strategies for minimizing negative effects of stressful stimuli in livestock, and particularly in reducing stress response, and improving behavior, immune function, morbidity, performance, and carcass characteristics before, during and/or after exposure to a stressful event. As demonstrated herein, short-term administration of cannabinoids from industrial hemp results in the altered behavior and stress response of cattle. Industrial hemp is defined by the USDA as Cannabis sativa with <0.3% tetrahydrocannabinol (THC). The cannabinoids can be used as a pre-transport and/or on arrival therapeutic for animals entering a feedlot to improve disease outcomes of bovine respiratory disease (BRD), biomarkers of stress, performance characteristics, and carcass traits at harvest in cattle and other livestock. As demonstrated in the working examples, short-term administration of cannabinoids from industrial hemp reduces weight loss in treated animals, results in lower cortisol levels in the treated animals when off-loading from transport, and gives the animals a higher mechanical nociception threshold (MNT), which indicates a better tolerance to pain stimulus.

The present disclosure is broadly concerned with methods of improving performance, health, and well-being in animals, particularly in ruminant or pre-ruminant animals (e.g., cattle, sheep, goats, llamas, etc.). The methods comprise administering to the animal an effective amount of a cannabinoid (and preferably a combination of cannabinoids) before, during, and/or after subjecting the animal to stressful event, such as transportation, weaning, calving, regrouping, comingling, dehorning, disbudding, castration, branding, tagging, microchipping, vaccination, and other animal management procedures collectively known as processing. Advantageously, the animal has improved performance (such as weight gain or reduced weight loss, and overall health and well-being) after the stressful event as compared to a control.

The disclosure also provides a veterinary formulation for improving performance in an animal before, during, and/or after a stressful event. The veterinary formulation could be useful for reducing weight loss and improving health and resilience in a ruminant or pre-ruminant animal following a stressful event. In one or more embodiments, the formulation is an animal feed or animal feed additive comprising an effective amount of a cannabinoid (and preferably a combination of cannabinoids).

Combinations of cannabinoids are particularly preferred, such as cannabidiolic acid (CBDA), cannabidivarinic acid (CBDVA), cannabidiol (CBD), cannabichromenic acid (CBCA), cannabigerolic acid (CBGA), tetrahydrocannabinolic acid A (THCA-A), and combinations thereof, with CBDA being the predominantly preferred cannabinoid. In one or more embodiments, naturally sourced combinations of cannabinoids (e.g., industrial hemp) are used. Although the mechanism of action is not fully understood, it is recognized that interactions between multiple cannabinoids act in synergy to achieve an enhanced effect than would be achieved with the individual compounds. It is envisioned that the effective amount of cannabinoid may be provided in the feed via a natural source of cannabinoids, such as industrial hemp, which may be mixed with animal feed. It is further envisioned that the cannabinoid actives may be distilled, isolated, and/or concentrated and applied as a top dressing to animal feed, oral drench, bolus, capsule, injection or transdermal formulation in an effective amount to achieve the desired effect. As used herein, an “effective amount” refers to an amount capable of providing bioavailable levels of the active compound (i.e., cannabinoid) sufficient to achieve the desired performance improvement. In one or more embodiments, an effective target amount of cannabinoid is up to 30 mg/kg bodyweight of the animal, and as low as 0.5 mg/kg bodyweight of the animal. It will be appreciated that the amount of a natural source of cannabinoids may need to be adjusted depending upon its relative cannabinoid content. The cannabinoids can be further formulated with taste enhancers, such as molasses, corn syrup, distillers' grains, protein or carbohydrate supplements including corn, beet pulp, soy, wheat, sorghum or other grains or grain byproducts and the like.

In one or more embodiments, the cannabinoid(s) may be administered at the time of the stressful procedure or at any time during the 24 hours before or after the stressful event. In one or more embodiments, the cannabinoid(s) are administered to the animal daily for at least 5 days, preferably at least 7 days, more preferably from 5-7 days before the animal is exposed to the stressful event. In one or more embodiments, the cannabinoid(s) are administered to the animal immediately after the stressful event (i.e., within 30 minutes to 1 hour after the stressful event), and thereafter daily for up to 5 days, preferably up to 7 days, preferably 5-7 days after the event. Preferably, in either instance, the cannabinoid(s) are administered daily for no more than a total time period of two weeks, preferably no more than 10 days, even more preferably no longer than 1 week. As demonstrated in the examples, a plasma steady-state of cannabinoid concentrations in the treated animals can be achieved at approximately 7 days of daily administration.

In one or more embodiments, the cannabinoid(s) may be distilled, isolated and/or concentrated. In one or more embodiments, the cannabinoid(s) may be administered as a single loading dose, a sustained release formulation, or multiple daily doses. In one or more embodiments, the cannabinoid(s) may be administered orally, topically, transdermally, or by injection to the animal.

Although not necessary, the cannabinoid(s) can be administered in combination with other recognized therapeutics or anti-inflammatory compounds, such as cyclooxygenase (COX) enzyme inhibitors, prostaglandin receptor antagonists and local anesthetics including, but not limited to, aspirin, meloxicam, firocoxib, flunixin, carprofen, ketoprofen, tolfenamic acid, sodium salicylate, grapiprant, lidocaine, procaine and bupivacaine, to further minimize the negative effects of the stressful event on the animal.

Advantageously, these novel approaches for animal management and handling are effective for improving performance and reducing disease without the use of anesthetics, opioids, or other adjunctive therapy (e.g., antibiotic, hormonal implant, ionophore, other growth promotants, or vaccines).

In preferred embodiments, the invention relates to cattle or other livestock, and specifically improving the performance of ruminant and pre-ruminant bovines. The term “pre-ruminant” refers to young animals before development of a functional ruminant stomach compartment. The term “bovine” is used herein to refer generally to all types of domesticated cattle (bovines), including heifers (young females), bulls (uncastrated males), steers (castrated males), calves (young cattle of both sexes), and cows (adult females), unless otherwise specified. The phrase “improving performance,” as used herein encompasses any suitable marker of performance and well-being of the animal, including behavior such as time spent lying down, time spent standing or walking, and weight-bearing, as well as biomarkers such as weight gain and carcass performance, incidence of disease, cortisol levels, PGE₂ levels, body temperature, and the like. The inventive protocols can reduce cortisol and PGE₂ levels in livestock, as well as increase resting time. As such, the compositions and methods, if administered before transport or immediately upon arrival, reduce the impact of long-distance transportation on circulating physiological biomarkers of stress and inflammation in the animal. The approach can also be used to mitigate stressful experiences such as weaning, transportation, processing, comingling, and regrouping. Each of the foregoing parameters is judged in comparison to a control, where the “control” is an animal of a similar age, weight, condition, etc. that has been subjected to the same stressful event, but without having received cannabinoids before, during, and/or after the event.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

Short-term feeding of industrial hemp: plasma cannabinoid concentrations, behavior outcomes, and immune modulation.

### Abstract

Industrial hemp (IH) is defined as Cannabis sativa containing 0.3% Tetrahydrocannabinol (THC) was legalized in the 2018 farms bill. The impact of cannabinoids in IH, especially after repeat exposure has not been investigated. Sixteen male castrated Holstein cattle weighting (±SD) 447±68 kg were enrolled onto the study. Cattle were allocated into two treatment groups either receiving IH (HEMP, n=8) or a control (CNTL, n=8). Cattle in the HEMP group were fed 25 g IH mixed in 200 g of grain once a day for 14 days to target a daily dose of 5.5 mg/kg of cannabidiolic acid (CBDA). Blood sample were taken at predetermined time points for plasma cannabinoid, serum cortisol, serum haptoglobin, liver enzymes, serum amyloid A, and prostaglandin E2 concentrations. Cattle had activity monitors placed 96 hours prior to the first IH feeding and for 96 hours after the final hemp feeding. Cortisol levels in the HEMP group were lower than the CNTL group (P=0.001). Cattle in the HEMP group had lower PGE₂ concentrations and a −8.8% decrease from baseline was observed. Whereas the CNTL group had 10.2% increase from baseline for PGE₂. No differences for haptoglobin or serum amyloid A were observed. These results show IH feeding and CBDA alters behavior as well as reduces the stress response and PGE₂ levels.

### Introduction

With the legalization of industrial hemp [Cannabis sativa containing<0.3% Tetrahydrocannabinol (THC)] in the 2018 United States Farm Bills interest in IH has grown. There are numerous uses for IH and its various plant components. Additionally, there is interest and potential for the inclusion of IH and IH by-products in animal feeds. The nutritional content, digestibility, and cannabinoid concentrations of various IH plant components have been described. This data shows various IH plant components may be suitable for inclusion into cattle rations.

The pharmacokinetics of cannabinoids, specifically cannabidiolic acid (CBDA), in cattle following a single dose of IH flowers has also been previously described. However, there is no data regarding the effects of cannabinoids on cattle health and behavior in the literature. If hemp is to be utilized as an ingredient in the ration of cattle, knowing the pharmacokinetics and potential biological effects of cattle exposed to repeated doses of IH is prudent. This requisite data is needed if IH and IH by-products are to be considered by the US Food and Drug Administration (FDA) and the Association of American Feed Control Officials (AAFCO). The objectives of this study were to: 1) determine the plasma concentrations and pharmacokinetics of cannabinoids during a 14-day feeding period; and 2) impacts of feeding IH on blood inflammatory biomarkers and activity.

### Results

### Plasma Cannabinoid Concentrations and Pharmacokinetics

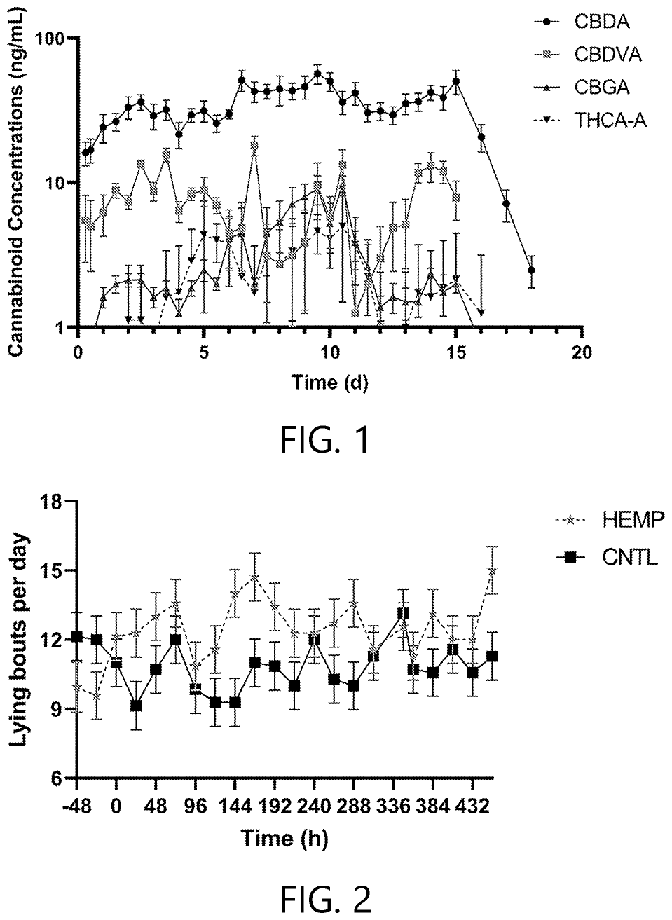

Of the 16 cannabinoids included in the analysis, 10 were below their level of detection at all time points. Cannabinoids detected were cannabidiolic acid (CBDA), cannabidivarinic acid (CBDVA), cannabidiol (CBD), cannabichromenic acid (CBCA), cannabigerolic acid (CBGA), and tetrahydrocannabinolic acid A (THCA-A). Plasma CBDA concentrations over time are shown in FIG. 1. Final cannabinoid doses on a milligram per kilogram basis are in Table 1. The pharmacokinetic parameters of CBDA for all HEMP cattle are summarized in Table 2. In the initial hemp feeding period, the geometric mean maximum concentration of CBDA was 22.1 ng/ml and the geometric mean minimum concentration of CBDA was 12.3 ng/ml. These were observed at 16.7 h and 9.3 h after the initial IH feeding respectively. The geometric mean of the terminal half-life (T½) was 15.3 h. A geometric mean residence time of 822.2 h was determined, and the geometric mean AUC_{0-∞} was 13,569.6 h×ng/mL. The mean accumulation index was 1.5 for the 14-day feeding period. No cannabinoids were detected in the plasma of the CNTL cattle.

TABLE 1
Individual animal weights (kg) and doses (mg) of individual
cannabinoids administered to each animal in the HEMP group

| | Animal ID | | | | | | | | Mean |
|---|---|---|---|---|---|---|---|---|---|
| | 1 | 2 | 3 | 4 | 5 | 6 | 7 | 8 | (SD) |
| Body weight, kg | 479.5 | 513.6 | 465.9 | 464.1 | 634.1 | 420.9 | 504.5 | 472.7 | 494.4 |
| | | | | | | | | | (59.0) |
| Cannabinoid doses, | | | | | | | | | |
| mg/kg | | | | | | | | | |
| Cannabidiolic acid | 5.7 | 5.3 | 5.9 | 5.9 | 4.3 | 6.5 | 5.4 | 5.8 | 5.6 |
| (CBDA) | | | | | | | | | (0.6) |
| Cannabidivarinic acid | 0.005 | 0.005 | 0.005 | 0.005 | 0.004 | 0.006 | 0.005 | 0.005 | 0.005 |
| (CBDVA) | | | | | | | | | (0.000) |
| Tetrahydrocannabinolic | 0.12 | 0.11 | 0.12 | 0.12 | 0.09 | 0.14 | 0.11 | 0.12 | 0.12 |
| acid-A (THCA-A) | | | | | | | | | (0.01) |
| Cannabichromenic acid | 0.15 | 0.14 | 0.16 | 0.16 | 0.11 | 0.17 | 0.14 | 0.15 | 0.15 |
| (CBCA) | | | | | | | | | (0.02) |
| Cannabidiol (CBD) | 0.18 | 0.17 | 0.19 | 0.19 | 0.14 | 0.21 | 0.17 | 0.19 | 0.18 |
| | | | | | | | | | (0.02) |
| 9- | 0.05 | 0.05 | 0.05 | 0.05 | 0.04 | 0.06 | 0.05 | 0.05 | 0.05 |
| Tetrahydrocannabinol | | | | | | | | | (0.01) |
| (9-THC) | | | | | | | | | |
| Cannabigerolic acid | 0.19 | 0.18 | 0.20 | 0.20 | 0.15 | 0.22 | 0.18 | 0.20 | 0.19 |
| (CBGA) | | | | | | | | | (0.02) |
| Cannabigerol (CBG) | 0.012 | 0.011 | 0.012 | 0.012 | 0.009 | 0.014 | 0.011 | 0.012 | 0.01 |
| | | | | | | | | | (0.00) |
| Cannabichromene | 0.07 | 0.06 | 0.07 | 0.07 | 0.05 | 0.08 | 0.06 | 0.07 | 0.07 |
| (CBC) | | | | | | | | | (0.01) |
| Cannabinol (CBN) | ND¹ | ND | ND | ND | ND | ND | ND | ND | |
| Cannabidivarin | ND | ND | ND | ND | ND | ND | ND | ND | |
| (CBDV) | | | | | | | | | |

¹ND—Cannabinoids not detected in IH

TABLE 2
Pharmacokinetic parameters of cannabidiolic
acid (CBDA) following feeding industrial
hemp for 14 days at a target dose of 5.5 mg/kg (n = 8).

| | | Geometric | | |
|---|---|---|---|---|
| Parameter | Unit | Mean | Median | Range |
| λ_{z} | 1/h | 0.05 | 0.05 | 0.04-0.06 |
| T1/2 | h | 15.3 | 14.8 | 11.3-19.8 |
| Tmax | h | 16.7 | 24.0 | 8.0-24.0 |
| Cmax | ng/ml | 22.1 | 22.2 | 12.4-57.9 |
| AUC_{0-∞} | h × ng/ml | 13569.6 | 13595.5 | 8888.6-25564.7 |
| AUC₀₋ₗₐₛₜ | h × ng/ml | 13508.4 | 13542.7 | 8819.2-25465.8 |
| AUC | % | 0.38 | 0.49 | 0.15-0.78 |
| extrapolated | | | | |
| MRT_{0-∞} | h | 822.2 | 771.5 | 573.2-1424.8 |
| Accumulation | | 1.52 | 1.49 | 1.30-1.76 |

### Blood Inflammatory Biomarkers

All results are presented as mean±SEM unless otherwise noted. Inflammatory biomarker summary data are presented in Table 3.

TABLE 3
Summary of mean (95% confidence interval) cortisol and
prostaglandin E₂ (PGE₂) concentrations for cattle fed industrial
hemp once a day for 14 days (HEMP) or control cattle (CNTL).

| | | | P-value | | |
|---|---|---|---|---|---|
| | | | | | Treat- |
| | | | | | ment |
| | HEMP | CNTL | Treat- | | × |
| Parameter | (n = 8) | (n = 8) | ment | Time | time |
| Cortisol, ng/ml | 1.59 | 5.97 | 0.001 | 0.58 | 0.12 |
| | (−0.22-3.40) | (4.43-7.50) | | | |
| PGE₂, pg/mL | 42.6 | 48.2 | 0.09 | 0.053 | 0.88 |
| | (38.0-47.2) | (43.6-52.8) | | | |
| PGE₂ percent | −8.8% | 10.2% | 0.03 | 0.08 | 0.51 |
| change from | (−20.7-3.0%) | (−1.7-22.1%) | | | |
| baseline | | | | | |
| Haptoglobin, | 8.66 | 8.63 | 0.93 | 0.61 | 0.94 |
| mg/dL | (8.14-9.17) | (8.11-9.17) | | | |
| Serum Amyloid | 12.65 | 8.91 | 0.29 | 0.0007 | 0.0008 |
| A, μg/mL | (7.80-20.53) | (5.48-14.46) | | | |

### Cortisol

Cattle in the HEMP group had lower mean cortisol concentrations than the CNTL cattle (P=0.0013). These differences were most notable on day 14 and day 19. On day 14, HEMP cattle had mean cortisol concentrations of 0.76±0.32 ng/mL and the CNTL had a mean cortisol concentration of 6.57±0.93 ng/mL (P=0.002). Similarly on day 19 the HEMP cattle had mean cortisol concentrations of 2.75±1.14 ng/ml and the CNTL had mean concentrations of 7.90±1.28 ng/mL (P=0.004).

### Ex Vivo Prostaglandin E₂

There tended to be treatment differences observed between treatment groups for PGE₂ concentrations (P=0.09). Calves in the HEMP group had mean PGE₂ concentrations of 46.5±3.9 pg/mL, 42.4±5.2 pg/mL, 46.0±5.0 pg/mL, and 33.2±2.0 pg/mL for the baseline, day 7, day 14, and day 19 time points. Calves in the CNTL group had mean PGE₂ concentrations of 48.8±8.1 pg/mL, 51.0±9.5 pg/mL, 53.5±2.7 pg/mL, and 42.0±2.8 pg/mL for the baseline, day 7, day 14, and day 19 time points.

There were differences in the percent change of PGE₂ from baseline between groups (P=0.03). Calves in the HEMP group had mean percent changes in baseline of −6.25±13.2%, −0.3±9.0% and −24.4±8.8% for days 7, 14, and 19 respectively. Calves in the CNTL group had mean percent changes in baseline of 11.9±16.7%, 24.6±14.3% and −0.18±14.3% for days 7, 14, and 19.

### Haptoglobin

No differences in haptoglobin were observed between HEMP and CNTL cattle. Mean haptoglobin concentrations were 8.66±0.14 mg/dl and 8.63±0.19 mg/dL for the HEMP and CNTL cattle respectively (P=0.93). Haptoglobin concentrations did not change over time (P=0.61).

### Serum Amyloid A (SAA)

A significant treatment by time interaction was observed. Cattle in the HEMP group had elevated SAA concentrations compared to the CNTL group at 7 d (53.0±1.4 μg/mL vs. 8.2±1.4 μg/mL (P=0.0065)). Mean SAA concentration were not different between groups at other time points.

### Hepatic Enzyme Concentrations

Mean hepatic enzyme concentrations are presented in Table 4. All samples for total protein, albumin, globulin, aspartate transaminase (AST), gamma glutamyltrasferase (GGT) were within the reference intervals for the reporting diagnostic laboratory (KSVDL). All animals in both groups had elevated alkaline phosphatase (ALP) at all time points. There were no significant treatment effects (P=0.87) or treatment by time interactions (P=0.44) observed between ALP between groups. Significant differences for AST were observed with mean CNTL levels of 80.4±3.1 U/L compared to HEMP at 69.8±3.1 U/L (P=0.03). A treatment difference for GGT was observed. The mean CNTL GGT levels (11.7±0.8 U/L) were lower than the GGT levels of the HEMP cattle (14.1±0.8 U/L; P=0.05).

Sorbitol dehydrogenase (SDH) levels tended to be higher in CNTL cattle (16.7±0.7 U/L) compared to HEMP cattle (14.7±0.7 U/1) (P=0.08). There was a significant treatment by time interaction observed where the CNTL cattle mean SDH level increased from 15.5 U/L prior to study commencement to 17.3 U/L on day 19. The HEMP cattle mean SDH levels decreased from 15.7 U/L prior to IH feeding to 13.6 U/L on day 19 (P=0.04). All mean levels were below the upper range of the reference interval for the reporting laboratory. However, there were 11 sample above the upper end of the laboratory's reference range of 18.4 U/L.

TABLE 4
Summary of mean (95% Confidence intervals) hepatic specific
serum blood chemistry profiles prior to industrial hemp feeding
and at 7, 14, and 19 days post feeding for cattle fed
industrial hemp once a day for 14 days (HEMP)
or control cattle (CNTL).

| | HEMP | | | | P-value |
|---|---|---|---|---|---|
| Parameter | n = 8 | | | | Treat- |
| (Ref range) | 0 | 7 | 14 | 19 | ment |
| Total protein | 6.3 | 6.7 | 6.7 | 6.6 | 0.60 |
| (6.0-9.0 g/dL) | (6.1-6.5) | (6.5-6.9) | (6.5-6.9) | (6.4-6.8) | |
| Albumin | 3.4 | 3.5 | 3.6 | 3.6 | 0.33 |
| (3.1-4.3 g/dL) | (3.3-3.5) | (3.3-3.6) | (3.4-3.7) | (3.4-3.7) | |
| Globulin, g/dL | 2.9 | 3.2 | 3.1 | 3.1 | 0.27 |
| (n/a¹) | (2.7-3.1) | (3.0-3.4) | (2.9-3.3) | (2.9-3.3) | |
| AST² | 71.5^{a,b} | 68.1^{b} | 70.5^{a,b} | 68.9^{b} | 0.03 |
| (53-156 U/L) | (64.5-78.5) | (61.1-75.1) | (63.5-77.5) | (61.9-75.9) | |
| ALP | 189.6 | 178.3 | 163.3 | 165.9 | 0.87 |
| (21-81 U/L) | (145.1- | (133.7- | (118.7- | (121.4- | |
| | 234.1) | 222.8) | 207.8) | 210.4) | |
| GGT | 14.4^{a,b} | 14.9ª | 14.0^{a,b} | 13.3^{a-c} | 0.05 |
| (10-39 U/L) | (12.4-16.4) | (12.9-16.9) | (12.0-16.0) | (11.3-15.2) | |
| SDH | 15.7^{a,b} | 14.3ª | 15.1^{a,b} | 13.6ª | 0.08 |
| (6.1-18.4 U/L) | (13.6-17.7) | (12.2-16.3) | (13.0-17.1) | (11.5-15.6) | |
| | CNTL | | | | P-value |
| Parameter | n = 8 | | | | Treat- |
| (Ref range) | 0 | 7 | 14 | 19 | ment |
| Total protein | 6.4 | 6.6 | 6.7 | 6.8 | 0.60 |
| (6.0-9.0 g/dL) | (6.2-6.7) | (6.4-6.8) | (6.5-6.9) | (6.6-7.0) | |
| Albumin | 3.3 | 3.4 | 3.5 | 3.5 | 0.33 |
| (3.1-4.3 g/dL) | (3.1-3.4) | (3.3-3.5) | (3.4-3.6) | (3.4-3.5) | |
| Globulin, g/dL | 3.2 | 3.2 | 3.3 | 3.3 | 0.27 |
| (n/a¹) | (3.0-3.4) | (3.0-3.3) | (3.1-3.4) | (3.1-3.4) | |
| AST² | 82.8^{c} | 79.9^{a,c} | 79.0^{a,c} | 80.0^{a,c} | 0.03 |
| (53-156 U/L) | (75.8-89.7) | (72.9-86.9) | (72.0-86.0) | (73.0-87.0) | |
| ALP | 180.1 | 188.0 | 175.3 | 173.0 | 0.87 |
| (21-81 U/L) | (135.6- | (143.5- | (130.7- | (128.5- | |
| | 224.6) | 232.5) | 219.8) | 217.5) | |
| GGT | 11.0^{c} | 11.6^{b,c} | 12.1ª^{-c} | 12.0^{b,c} | 0.05 |
| (10-39 U/L) | (9.0-13.0) | (9.6-13.6) | (10.1-14.1) | (10.0-14.0) | |
| SDH | 15.5^{a,b} | 17.6^{b} | 16.3^{a,b} | 17.3^{b} | 0.08 |
| (6.1-18.4 U/L) | (13.4-17.5) | (15.6-19.7) | (14.3-18.4) | (15.3-19.4) | |

^{a,b,c}Different letter within rows are significantly different (P < 0.05)
¹n/a; calculated value. No laboratory reference given.
²AST, aspartate transaminase; ALP, alkaline phosphatase; GGT, gamma glutamyltrasferase; SDH, sorbitol dehydrogenase

### Activity Monitoring

Activity data is summarized in Table 5. Cattle in the HEMP group took a mean 1552 steps per day compared to the 1547 steps per day for the CNTL cattle (P=0.97). A time effect was observed (P<0.0001) as well as a treatment by time interaction (P<0.0001). Despite the differences in step count, cattle in the HEMP group spent more time lying down compared to the CNTL cattle. Cattle in the HEMP group spent a mean 14.1 h/d (95% CI: 13.6-14.6 h/d) lying compared to the 13.4 h/d (95% CI: 12.9-138. h/d) for the CNTL cattle (P=0.03). Cattle in the HEMP group had more lying bouts per day following IH administration (FIG. 2) at 12.4 bouts/d (95% CI: 11.0-13.9 bouts/d) compared to 10.9 bouts/d (95% CI: 9.4-12.2 bouts/d; P=0.12). A treatment by time interaction was observed as the HEMP cattle had few lying bouts prior to IH feeding and then increasing bouts/d after IH feeding. A lower motion index in the HEMP cattle was observed over time (P<0.0001).

TABLE 5
Summary of mean (95% confidence interval) accelerometer data
over 24 hour intervals for cattle fed industrial hemp once a
day for 14 days (HEMP) or control cattle CNTL).

| | | | P-value | | |
|---|---|---|---|---|---|
| | | | | | Treat- |
| | | | | | ment |
| | HEMP | CNTL | | | × |
| Parameter | (n = 8) | (n = 8) | Treatment | Time | time |
| Steps | 1552.2 | 1547.2 | 0.97 | <0.0001 | <0.0001 |
| | (1367.9- | (1362.9- | | | |
| | 1736.5) | 1731.5) | | | |
| Standing time, | 9.9 | 10.6 | 0.03 | <0.0001 | <0.0001 |
| h/d | (9.4-10.4) | (10.2-11.1) | | | |
| Lying time, h/d | 14.1 | 13.4 | 0.03 | <0.0001 | <0.0001 |
| | (13.6-14.6) | (12.9-13.8) | | | |
| Lying bouts, #/d | 12.4 | 10.9 | 0.12 | 0.01 | 0.0003 |
| | (11.0-13.9) | (9.4-12.3) | | | |
| Motion index | 7099.6 | 7222.3 | 0.83 | <0.0001 | <0.0001 |
| | (6256.8- | (6379.6- | | | |
| | 7942.4) | 8065.1) | | | |

### DISCUSSION

This is the first report of repeated IH administration to cattle and impact on activity behavior and selected blood parameters. The data here shows cannabinoid administration by daily feeding of IH impacts behavior and the stress response in cattle. These findings are important as IH may present a viable way to mitigate stressful experiences such as transportation and regrouping of cattle.

Cattle in the HEMP group were individually hand fed IH daily as part of their grain ration to ensure complete intake. At each feeding, cattle were monitored by researchers to ensure the complete IH dose was consumed. To prevent sorting, the IH was chopped for better mixing with the grain, and molasses was applied just prior to feeding. The amount of IH fed was based on the mean body weight of the animals in the HEMP group obtained on the day of randomization and activity monitor attachment. All cattle received the same amount of IH to replicate a group feeding rate.

No cannabinoids were detected in any of the CNTL cattle samples. A similar group of cannabinoids were detected in the plasma of the HEMP cattle as previously reported with CBDA being the predominate cannabinoid in fresh IH plant components. A similar terminal half-life of 15 h was observed and it is comparable to those previously published of 14 h. The maximum CBDA concentrations (Cmax) following the first dose were lower than those previously reported (22 ng/mL vs 72.7 ng/mL). Time to reach maximum concentrations after the first dose were longer than those published. These differences highlight that there is additional work needed to better understand the absorption, distribution, metabolism and excretion of cannabinoids in cattle.

Based on the accumulation ratio of CBDA in this study, there is evidence of drug accumulation. The mean accumulation ratio was 1.52 with a range of 1.30 to 1.76. This indicates concentrations at steady-state were approximately 1.5-times the mean concentrations during the first dosing period (24 hours). Further work is needed to determine if this accumulation is clinically relevant. Based on visual inspection of the data, plasma CBDA steady state concentrations were reached at approximately 7 days of IH administration.

The cannabinoids CBDVA, CBGA, and THCA-A were detected in the plasma of all cattle. The doses for each were 0.005 mg/kg for CBDVA, 0.2 mg/kg for CBGA, and 0.1 mg/kg for THCA-A. Despite being present in a relatively small doses of 0.005 mg/kg in the current study and 0.02 mg/kg in an earlier study; CBDVA is found in relatively higher concentrations than other cannabinoids. Cannabidiol (CBD) was detected in 159 of the 288 samples from IH cattle, and CBCA was detected in 93 of 288 samples from HEMP cattle. The impact the rumen environment may have on cannabinoids is unclear and deserves further investigation.

Differences in the doses of cannabinoids other than CBDA are due to variation in the cannabinoid content of IH. The variety used in the current study was “Otto Stout” and the cannabinoid profile was different than the variety used in an earlier study. A representative sample was used to analyze the IH for cannabinoid content, but variation within the lot cannot be ruled out. This poses an area of concern if IH were to be legalized as an animal feed ingredient.

Cattle in the HEMP group had lower PGE₂ levels compared to CNTL cattle. Additionally, a decrease from baseline for PGE₂ was observed for cattle in the HEMP group and compared to the increase in PGE₂ from baseline for cattle in the CNTL group. These findings suggest a cannabinoid linked reduction in PGE₂ expression. CBDA has been previously shown to exhibit COX-2 inhibitory properties. Additionally, CBDA has been shown to have anti-inflammatory and anti-hyperalgesia effects in rats with carrageenan induce inflammation. It has been suggested that the phenolic ring in CBDA's structure may mimic salicylic acid. Further investigation is needed to determine if cannabinoid the reduction in PGE₂ is clinically relevant in cattle.

There were observed differences in hepatic specific serum biochemistry parameters. Hepatic enzymes were evaluated as evidence suggests cannabinoids are metabolized by liver microsomes. Although differences in hepatic enzymes were seen between the HEMP and CNTL cattle; most parameters were within the reference range of the laboratory (KSVDL). Alkaline phosphatase (ALP) was elevated for all cattle and is most like due to contribution of the bone ALP isoform prominent in growing animals.

Feeding IH caused an increase in SAA concentrations 7 d in the present study in cattle in the HEMP group. The impact IH and/or cannabinoids had in these increases is not clear as there is no veterinary literature. Although ALP was elevated in all cattle, other hepatic specific enzymes measured (SDH and GGT) were within normal reporting limits for the lab. Furthermore, the role IH and cannabinoids may have in mitigating SAA production following an inflammatory event in cattle is not known and deserves further research.

Cortisol concentrations were lower in the HEMP cattle compared to CNTL cattle. This is interesting as both groups were managed the same, but the HEMP group had twice daily venipunctures to obtain blood for cannabinoid concentrations. Synthetic cannabinoid receptor agonists given intravenously provoked an increase in serum cortisol, but hypoalgesia to cutaneous pain and thermal stimuli. Cannabinoids, specifically cannabidiol, have been shown to reduce stress and anxiety in mice. Conversely, CBD did not lower cortisol in dogs following a simulated fireworks model.

Accelerometers have been shown to be an accurate measure of cattle behavior. Raw accelerometer data was condensed to a 24 hour period to remove the diurnal effect of cattle's natural behavior. There were significant differences in the activity of HEMP and CNTL cattle. Most notably were the change in lying time and lying bouts. Cattle in the HEMP group had fewer lying bouts prior to the start of IH feeding and increased lying time after IH feeding started. Lying behavior has been associated with cattle welfare with longer lying time indicating better welfare.

These results, coupled with the cortisol data, suggest cattle fed cannabinoids via IH have lower stress biomarkers and improved lying times. Further work is needed to determine if cannabinoids can alter the stress response in cattle during stressful times such as transportation and weaning

### Material and Methods

### Ethics Statement

All experimental procedures were approved by the Institutional Animal Care and Use Committee at Kansas State University (IACUC #4421). Industrial hemp was grown and handled under license of the Kansas Department of Agriculture Industrial Hemp Research Program (licenses numbers: KDA-0621466839 and KDA-0302873296).

### Subjects and Housing

Sixteen (16) male castrated Holstein cattle weighting (±SD) 447±68 kg were enrolled onto the study. Cattle had been acclimated at the research facility and halter trained prior to study commencement. Cattle were group housed in outdoor dirt pens with access to shelter. Pen size per calf exceeded the recommendations set forth in the Guide for the Care and Use of Agricultural Animals in Research and Teaching. Calves had ad libitum access to grass hay and water via self-filling trough. In addition to the research feed, calves were fed a custom grain mix at 8:00 and 16:00 h by animal care staff.

### Animal Groups

Cattle were assigned to one of two treatment groups. Treatment groups were:
1) HEMP (n=8)-cattle fed 25 g IH mixed in 200 g grain once a day for 14 days; or
2) CNTL (n=8)-control cattle fed grain only for 14 days.

### Industrial Hemp Dosing and Cattle Sampling

Cattle in the HEMP group were individually fed 25 g IH mixed in 200 g grain placed in rubber feed pans at 07:00 daily for 14 days. The IH was fed at 25 g per animal per day to target a mean dose of CBDA of 5.5 mg/kg. Daily IH feeding was after the daily blood sample for cattle in the HEMP group.

Cattle were blood sampled at predetermined time points from the jugular or coccygeal veins using a needle and vacutainer. Whole blood was collected into tubes containing sodium heparin, EDTA, sodium citrate, and no additives. Blood samples from the HEMP cattle were obtained prior to initial IH feeding (day −1) and 8 and 12 h post-initial IH feeding; then every 12 h (prior to the next IH feeding); then (starting 12 hours after the last IH feeding) every 24 hours for 5 samples or 108 h after the last IH feeding Cattle in the CNTL group were blood sampled −24 h prior to the initial IH feeding; and on day 7, 14 and 19 post-initial IH feeding.

### Plasma Cannabinoid Analysis

Plasma cannabinoid concentrations were determined using UPLC-MS methods. All solvents used such as methanol, acetonitrile, isopropanol, formic acid were LC-MS grade. Cannabinoids standards were purchased in individual solutions in methanol (Cerilliant Corporation, Round Rock, TX), including: (±)-cis-11-Nor-9-carboxy-A 9-tetrahydrocannabinol glucuronide (THC-glu), (±)-11-Hydroxy-Δ9-tetrahydrocannabinol (THC-OH), cannabidivarinic acid (CBDVA), cannabidivarin (CBDV), cannabidiol (CBD), cannabidiolic acid (CBDA), Δ9-Tetrahydrocannabinolic acid A (THCA-A), cannabigerolic acid (CBGA), cannabigerol (CBG), Δ9-tetrahydrocannabinol (9-THC), Δ8-tetrahydrocannabinol (8-THC), cannabichromene (CBC), Δ9-tetrahydrocannabivarin (THCV), cannabichromenic Acid (CBCA), cannabinol (CBN), (−)-11-nor-9-Carboxy-Δ9-tetrahydrocannabinol (THC-acid). Cannabinoid analogs used as internal standards were also purchased in solution in methanol at 100 μg/ml (Cerilliant Corporation, Round Rock, TX): (±)-cis-11-Nor-9-carboxy-Δ9-tetrahydrocannabinol glucuronide (THC-glu-d₃), Cannabidiol-d₃ (CBD-d₃), Δ9-Tetrahydrocannabinol-d₃ (9-THC-d₃), (±)-11-nor-9-Carboxy-D9-tetrahydrocannabinol-d₉ (THC-acid-d₉), (±)-11-Hydroxy-Δ9-tetrahydrocannabinol-d₃ (THC-OH-d₃), Cannabichromene-do (CBC-d₉). All cannabinoids standards were kept in the freezer at −20° C. On the day of analysis, plasma samples were allowed to thaw. Plasma (samples, quality controls or negative control plasma) were mixed with the internal standard mixture at 200 ng/mL (not added to the negative control sample) and acetonitrile with 0.1% formic acid to precipitate the proteins. The mixture was vortexed for 5 seconds and centrifuge for 5 minutes at 7,000 g. The supernatant was then diluted with ultra-pure 18 □□ water before clean-up. The sample was loaded on a solid phase extraction plate using positive pressure nitrogen. Each well was washed twice with 0.25 mL of a mixture of methanol-water (25:75). The compounds were eluted with two-25 μL aliquots of acetonitrile-methanol (90:10) and diluted with 50 μL of water before analysis.

Cannabinoid analysis was performed using an Acquity H UPLC and a TQ-S triple quadrupole mass spectrometer (Waters Corp., Milford, MA). The chromatographic separation was performed with a UPLC column (Eclipse Plus C18, Agilent Technologies, Santa Clara, CA) 100×2.1 mm, 1.8μ, heated at 55° C. The flow rate was set at 0.5 mL/min, the mobile phase consisted of a gradient of acetonitrile (B) and water containing 0.1% formic acid (A) as follow: 0 min: 60% B, 6.50 min: 86% B, 7.50 min-9 min: 100% B, 9.01 m in 12 min: 60% B. The total run time was 12 min. The injection volume was 5 μL. The data acquisition was performed by electrospray ionization in positive and negative mode using multiple reaction monitoring. Linear regression with a weighing factor of 1/X was used and accepted if the coefficient of correlation R² was >0.99. Calibration curves were linear from 0.1 to 100 ng/ml for all cannabinoids. The lower limit of detection, lower limit of quantification, intra-day precisions, inter-day precisions, and inter-day accuracies for each cannabinoid analyte are summarized in Table 6.

TABLE 6
Lower level of detection (LOD), lower limit of quantification
(LOQ), intra-day precisions, inter-day precisions, and
inter-day accuracies for each cannabinoid analyte analyzed
following oral administration of industrial hemp.

| | | | Intra-day Precision | | |
|---|---|---|---|---|---|
| | | | (n = 3) | | |
| Cannabinoid analytes | LOD¹ | LOQ² | 1.75 | 47.5 | 95.0 |
| Tetrahydocannabinol- | 0.2 | 0.5 | 1.5 | 3.0 | 4.5 |
| glucuronide (THC-glc) | | | | | |
| Cannabidivarinic acid | 0.2 | 1.0 | 12.3 | 7.8 | 14.7 |
| (CBDVA) | | | | | |
| Hydroxy- | 0.2 | 0.25 | 2.2 | 6.1 | 3.7 |
| tetrahydrocannabinol | | | | | |
| (THC-OH) | | | | | |
| Tetrahydrocannabinolic- | 0.2 | 0.5 | 5.7 | 2.4 | 4.0 |
| acid (THC-acid) | | | | | |
| Cannabidivarin (CBDV) | 0.2 | 0.25 | 4.3 | 2.6 | 4.0 |
| Cannabichromenic acid | 1.0 | 2.5 | 16.7 | 9.2 | 6.8 |
| (CBCA) | | | | | |
| Tetrahydrocannabivarin | 0.2 | 0.25 | 4.3 | 3.4 | 3.0 |
| (THCV) | | | | | |
| Cannabigerol (CBG) | 0.2 | 0.25 | 4.8 | 3.0 | 2.5 |
| Cannabidiol (CBD) | 0.2 | 0.5 | 8.4 | 3.5 | 4.2 |
| Cannabinol (CBN) | 0.2 | 0.5 | 1.5 | 3.4 | 3.6 |
| 9-Tetrahydrocannabinol | 0.2 | 0.5 | 4.0 | 3.1 | 4.1 |
| (9-THC) | | | | | |
| 8-Tetrahydrocannabinol | 0.2 | 0.5 | 6.3 | 4.0 | 3.6 |
| (8-THC) | | | | | |
| Cannabichromene | 0.2 | 0.5 | 3.8 | 2.4 | 2.1 |
| (CBC) | | | | | |
| Tetrahydrocannabinolic | 1.0 | 2.5 | 8.5 | 3.7 | 8.0 |
| acid-A (THCA-A) | | | | | |
| Cannabichromenic acid | 1.0 | 2.5 | 3.6 | 4.7 | 9.3 |
| (CBCA) | | | | | |

| | Inter-day Precision | | | Inter-day Accuracy | | |
|---|---|---|---|---|---|---|
| | (n = 6) | | | (n = 6) | | |
| Cannabinoid analytes | 1.75 | 47.5 | 95.0 | 1.75 | 47.5 | 95.0 |
| Tetrahydocannabinol- | 10.5 | 8.6 | 7.2 | 106 | 105 | 106 |
| glucuronide (THC-glc) | | | | | | |
| Cannabidivarinic acid | 32.5 | 33.8 | 33.6 | 121 | 115 | 119 |
| (CBDVA) | | | | | | |
| Hydroxy- | 7.8 | 5.7 | 5.8 | 110 | 103 | 103 |
| tetrahydrocannabinol | | | | | | |
| (THC-OH) | | | | | | |
| Tetrahydrocannabinolic- | 4.2 | 3.5 | 5.2 | 112 | 103 | 103 |
| acid (THC-acid) | | | | | | |
| Cannabidivarin (CBDV) | 6.8 | 8.5 | 6.6 | 105 | 99 | 99 |
| Cannabichromenic acid | 24.7 | 29.2 | 24.3 | 105 | 99 | 99 |
| (CBCA) | | | | | | |
| Tetrahydrocannabivarin | 4.5 | 5.8 | 6.0 | 105 | 110 | 99 |
| (THCV) | | | | | | |
| Cannabigerol (CBG) | 8.3 | 10.5 | 12.0 | 107 | 100 | 95 |
| Cannabidiol (CBD) | 6.9 | 4.6 | 6.0 | 113 | 103 | 101 |
| Cannabinol (CBN) | 5.8 | 5.2 | 5.6 | 105 | 97 | 95 |
| 9-Tetrahydrocannabinol | 4.0 | 3.3 | 4.0 | 110 | 104 | 103 |
| (9-THC) | | | | | | |
| 8-Tetrahydrocannabinol | 5.3 | 5.5 | 5.3 | 104 | 100 | 101 |
| (8-THC) | | | | | | |
| Cannabichromene | 3.3 | 3.6 | 3.9 | 112 | 103 | 103 |
| (CBC) | | | | | | |
| Tetrahydrocannabinolic | 18.2 | 5.3 | 8.7 | 100 | 89 | 95 |
| acid-A (THCA-A) | | | | | | |
| Cannabichromenic acid | 20.3 | 7.3 | 10.9 | 113 | 100 | 108 |
| (CBCA) | | | | | | |

¹LOD, lower limit of detection
²LOQ, lower limit of quantification

### Cannabinoid Pharmacokinetic Analysis

Pharmacokinetic analysis for repeated dosing was performed on each HEMP animal to determine the pharmacokinetics of CBDA using non-compartmental methods using computer software (Phoenix® 8.2, Certara, Inc., Princeton, NJ, USA).

### Blood Inflammatory Biomarkers

### Cortisol

Serum cortisol was determined. Whole blood was collected from HEMP and CNTL cattle at −24 h, and 7, 14, and 19 d post-IH feeding for cortisol determination. Serum was separated off and placed into a cryovial for storage at −80° C. until analyzed.

Serum cortisol concentrations were determined using a commercially available radioimmunoassay (RIA) kit (MP Biomedicals, Irvine, CA, USA) following manufacturer specifications with minor modifications. The standard curve was extended to include 1 and 3 ng/ml by diluting the 10 and 30 ng/ml manufacturer-supplied standards 1:10 respectively. The assay had a detection range of 1 to 300 ng/mL. A low (25 ng/mL) and high (150 ng/ml) quality control (QC) were ran at the beginning and end of each set to determine inter-assay variability. Standard curves were plotted as a 4-parameter logistic curve with an R² of 0.998. Samples were analyzed in duplicate with those having a coefficient of variation (CV)>18% being re-analyzed.

Ex vivo Prostaglandin E₂

Ex vivo PGE₂ concentration were determined. Briefly, whole blood samples was collected from each animal −24 h prior to initial IH feeding, and 7, 14, and 19 d post-IH feeding. At each collection, whole blood (2 mL) samples were spiked with 20 μg lipopolysaccharide from Escherichia coli 0111: B4 (Sigma-Aldrich, MO, USA). Samples were incubated for 24 hours at 37 C. Samples were then centrifuged and the plasma was pipetted into individual cryovials and then stored at −80 C until analyzed. After thawing, plasma proteins were precipitated using methanol. Samples were then centrifuged at 3,000 g for 10 minutes and the PGE₂ concentration of the supernatant was determined using a commercially available enzyme-linked immunosorbent assay (ELISA) kit (Cayman Chemicals, MI, USA). The coefficient of variation for intra-assay variability was 10.7% and interassay variability was calculated as 10.8%.

### Haptoglobin and Hepatic Enzyme Concentrations

Serum was collected prior to hemp feeding and at 7, 14, and 19 d post-IH feeding. Serum samples were submitted to the Kansas State Veterinary Diagnostic Laboratory for biochemical analysis by photometric methods (Cobas 501, Roche Diagnostics, Indianapolis, IN, USA).

### Activity Monitoring

Cattle in both groups had daily activity monitored via commercially available accelerometers (IceQube, IceRobotics Ltd, South Queensferry, Edinburgh, Scotland UK). Accelerometers were placed on the left rear legs 96 h prior to the first hemp feeding. Accelerometer ID was paired with animal ID prior to placement and at the time of removal 5 days after the final IH feeding (day 19). Steps, time standing up, time lying down and lying bouts, and motion index data was collected via accelerometers at 15 minute intervals. Raw data was downloaded using a RFID reader and computer software (IceManager 2014, IceRobotics Ltd, South Queensferry, Edinburgh, Scotland UK).

Raw data analysis was performed. Steps, lying bouts and motion index were summated into 24-hour increments starting at 07:00 am on day −3 (72 h prior to first feeding) and ending at 7:00 am on day 19. Standing time and lying time were analyzed together due to their interrelation and summed on a 24 hour increment to account for the recording method of the accelerometer.

### Statistical Analysis

Cortisol concentrations were log transformed for normality prior to analysis. Statistical analysis was performed using computer software (JMP 15.0, SAS Inst. Inc., Cary, NC). Responses were analyzed using a mixed model with calf as the experimental unit. Treatment was assigned as the random effect; and time and treatment by time interaction were considered as fixed effects. Baseline PGE₂ concentrations were included as a fixed effect when analyzing the PGE₂ percent change from baseline. Post hoc tests were conducted using Tukey-Kramer adjustment. Statistical significance was set a P<0.05 a priori.

### Example 2

Impact of cannabinoids from industrial hemp on stress and immune biomarkers in cattle following long-distance transportation.

### Introduction

Cattle are moved in the U.S. for many reasons including: sale; placement into feedyards or rearing facilities; exposition/showing; and to abattoirs for harvest. Transportation has been shown to be a stressful event leading to increases in pro-inflammatory cytokines and increased morbidity and mortality. As shown in Example 1, cannabinoids from industrial hemp alter the behavior of cattle, specifically changes in lying and standing time.

The objective of this study is to determine the impact of cannabinoids from industrial hemp on stress biomarkers following transportation.

### Study Background

The vast majority of cattle in the United States undergo transportation at some point in their life. Cattle are transported from farm of origin to auction markets, feedlots, and finally to meat processing plants. Transportation has been shown to be a stressful event leading to increases in pro-inflammatory cytokines and increased morbidity and mortality. Thus, transportation has been shown to be a major risk associated with bovine respiratory disease.

Industrial hemp (IH) is defined as Cannabis sativa with <0.3% tetrahydrocannabinol (THC) in the 2014 Farm Bill. Its growth, cultivation, harvesting, and possession was made legal in the 2018 Farm Bill by removing IH as a DEA Schedule I drug. Industrial hemp is grown for three main uses: 1) fiber for textiles; 2) seeds for oil and protein; and 3) flowers for cannabinoid products such as cannabidiol (CBD) oil.

Our group has described the plasma cannabinoid concentrations of cattle exposed to IH and a known dose of cannabinoids. Cattle were administered a single oral dose of IH grown specifically for CBD production, and thus high in cannabinoid content. The predominant cannabinoid in raw IH is cannabidiolic acid (CBDA). Cannabidiolic acid is a precursor to CBD; and is converted by a decarboxylation reaction to CBD under heat and pressure. Following oral dosing of IH, CBDA and other cannabinoids are absorbed from the rumen and reach peak concentrations at 12-24 hours post-exposure.

As a follow-up, a group of Holstein cattle were fed IH once a day for 14 days at a target dose of 5.5 mg/kg CBDA. Cattle were hand-fed (individual feed pans) IH in the morning so complete intake could be assured. Additionally, cattle were co-housed with eight control cattle not fed IH. All cattle had accelerometers placed on their left rear leg to monitor activity and behavior. Additionally, blood samples were obtained from all cattle prior to the start of IH feeding as well as 7, 14, and 19 days for cortisol concentrations, prostaglandin E2 production, and acute phase protein (Serum Amyloid A and haptoglobin) levels. Cattle administered IH had lower cortisol concentrations, a decrease in prostaglandin E2 production, and changes in activity (unpublished data). These results show IH administration alters the stress response in cattle.

### Material and Methods

### Study Design

### Study Overview

Twelve (12) Holstein steers were acclimated for 7 days to ensure health status prior to study commencement. The study took place in four phases with all steers in each phase using a Latin square design. Steers were individually identified, weighed, evaluated for health status, and had an accelerometer placed on the left rear leg prior to enrollment. Steers were trained to walk across the gait analysis pressure mat. At 48 hours prior to start of the study (Day −2), steers were reweighed for randomization, accelerometer placement, and have baseline parameters taken. Baseline parameters include blood collection for cortisol, serum amyloid A, prostaglandin E metabolites, and CBC/biochemistry; MNT for pain sensitivity; infrared thermography (IRT) for ocular temperatures; and kinetic gait analysis via walking across the pressure mat system.

### Allotment/Randomization

Steers were weighted 24 hours prior to the start of the study and blocked by weight prior to random allocation into treatment groups. Randomization was accomplished using a random number generator (Microsoft Excel, Microsoft Corp).

### Treatment Groups

Steers were randomly assigned to an initial treatment group as described above. After a 14 day rest/washout period, each steer was enrolled into the next treatment. Treatments were administered so each steer was not transported more than once in a 28 day period. Treatments were administered just prior to loading the steer onto the trailer.

| | | Transportation | |
|---|---|---|---|
| | | Yes | No |
| Industrial | Yes | 3 (a) | 3 (b) |
| Hemp | No | 3 (c) | 3 (d) |

Group 1: (a) HEMP-TRANS→2 weeks→(d) PLBO-STAY→2 weeks→(c) PLBO-TRANS→2 weeks→(b) HEMP-STAY
Group 2: (c) PLBO-TRANS→2 weeks→(b) HEMP-STAY→2 weeks→(a) HEMP-TRANS→2 weeks→(d) PLBO-STAY
Group 3: (d) PLBO-STAY→2 weeks→(a) HEMP-TRANS→2 weeks→(b) HEMP-STAY->2 weeks→(c) PLBO-TRANS
Group 4: (b) HEMP-STAY→2 weeks→(c) PLBO-TRANS→2 weeks→(d) PLBO-STAY→2 weeks→(a) HEMP-TRANS

### Industrial Hemp Dosing

Industrial hemp was administered at a target dose of 5.5 mg/kg orally once prior to transportation. IH was placed into gelatin capsules prior to make an IH bolus. The weight of the IH bolus was recorded and used to determine the final cannabinoid dosing.

### Transportation

Cattle in the transport groups were loaded onto a trailer designed for cattle transport and driven round trip approximately 850 km. The duration of the trip was approximately 8 hours. Once unloaded, outcome variables were collected prior to cattle returning to their home pens. Steers were transported more than once per 30-day window.

### Outcome Variable Collection Procedures

### Body Weight Determination

Body weight data was collected each time the steer entered the chute using the chutes scale system (TruTest, Datamars, Mineral Wells, TX). Weights were recorded using paper records.

### Mechanical Nociception Threshold (MNT)

Mechanical nociception threshold is defined by a maximum force which induces a withdrawal response. Steers were restrained for mechanical nociception threshold determination. Using a hand-held pressure algometer (Wagner Instruments, Greenwich, CT), a force was applied perpendicularly at a rate of approximately 1 kg of force. Locations for MNT measures were at the level of the lateral coronary band at a point halfway between midline and the heel bulb of the left front and left rear feet. The 1 cm² rubber tip of the algometer were placed against the coronary band. A withdrawal response was indicated by an overt movement away from the applied pressure algometer. The MNT values were recorded by a second investigator to prevent bias by the investigator performing the MNT collection.

### Blood Sample Collection

Up to 20 ml of blood was collected in a syringe at each time point over the course of the study so that maximum blood collection was <7.5% of total blood volume. Given that the weight of the lightest steer was approximately 800 kg, the potential maximum blood collection volume over the study period is 3600 ml (800 kg×60 ml/kg=48,000 ml*0.075=3600 ml). Blood was collected from the jugular vein by venipuncture using a syringe or vacutainer and a 16 G needle.

### Blood Sample Processing and Tube Labeling

Whole blood samples were immediately transferred to tubes (Vacutainer®, BD Diagnostics) containing (1) no additive or; (2) EDTA anticoagulant. Blood samples were mixed by inverting the tube, labeled with a unique identifier, and immediately placed on ice. Blood samples were centrifuged for 10 minutes at 1,500 g. Collected plasma was placed in cryovials in duplicate with a single-use transfer pipette and frozen at −70° C. until analysis. The time of collection, processing, and freezing was recorded.

### Sample Analysis

### Cortisol

Cortisol concentrations were determined in duplicate via a radioimmune assay (Corti-Cote Cortisol RIA kit with ANS. Part no. 06α-256440 ICN; MP Biomedical, Santa Ana, CA, USA). The assay has a detection range of 0.0.01 to 150 ng/ml. Area under the effect curve (AUEC) was calculated using the linear trapezoidal method.

### Study Statistics

Responses were analyzed using mixed linear models where the steer was the experimental unit. Responses measured included body weight, cortisol, and MNT outcomes. All statistics were performed using statistical software (JMP).

### Results

### Body Weight

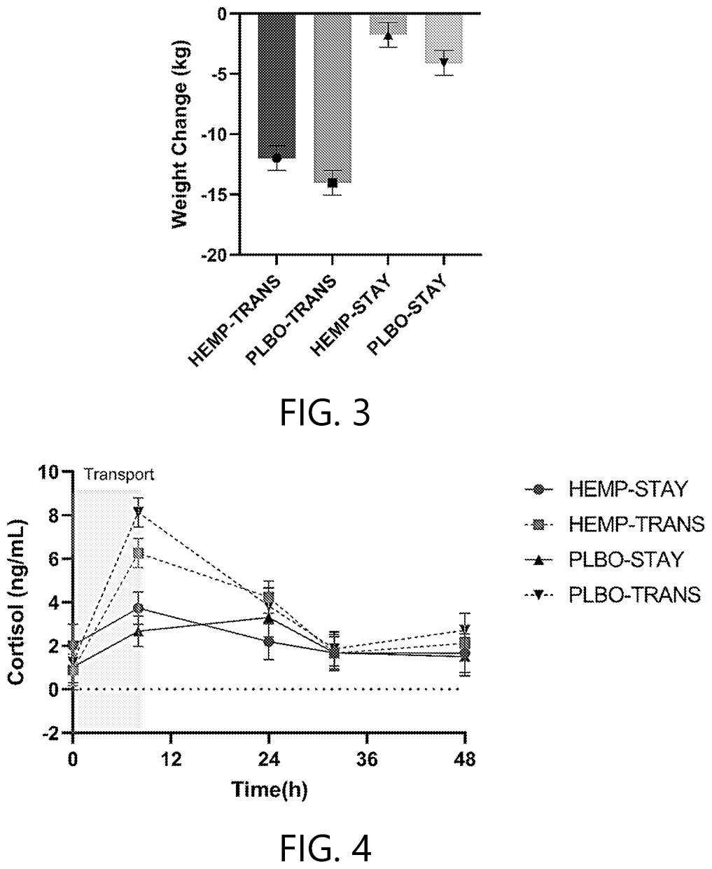

Steers in the HEMP groups had lower decreases in body weight compared to steers in the placebo PLBO groups. Weight changes (FIG. 3) were −1.76 kg (95% CI: 0.18-−3.77 kg), −4.09 kg (95% CI: −2.11-−6.07 kg), −11.98 kg (95% CI: −10.00-−13.95 kg), and −14.02 kg (95% CI: −12.05-−16.00 kg) for the HEMP-STAY, PLBO-STAY, HEMP-TRANS, and PLBO-TRANS groups respectively.

### Mechanical Nociception Threshold (MNT) Test

Steers administered HEMP had higher mean MNT measures compared to PLBO steers. The MNT measures for the treatment groups were: 5.59 kgf, 5.41 kgf, 5.43 kgf, and 5.30 kgf for the HEMP-STAY, HEMP-TRANS, PLBO-STAY, and PLBO-TRANS groups respectively.

### Cortisol

Steers in the HEMP-TRANS group had lower cortisol levels [6.27 ng/ml (95% CI: 4.94-7.60 ng/ml)] coming off the trailer (8 h; FIG. 4) compared to the PLBO-TRANS group [8.13 ng/ml (95% CI: 6.80-9.46 ng/mL)]. Additionally, steers in the HEMP-TRANS group had lower mean AUEC₀₋₂₄ values of 108.1 ng*h/mL compared to 132.2 ng*h/mL for the PLBO-TRANS group.

### Example 3

Impact of cannabinoids from industrial hemp on stress and immune biomarkers in cattle following long-distance transportation.

### Methods

### A. Study Animals

48 male Holstein/Holstein-cross calves will be enrolled. Calves will be between 8-10 weeks of age and weight approximately 70 kg. Cattle will be randomly allocated in one of three treatment groups:
1) IH-TRANS: Administered IH to a loading dose of 10.0 mg/kg cannabidiolic acid (CBDA) 12 h prior to transport and a second dose at 5.0 mg/kg before loading for transport and transported approximately 8 h (n=16 subjects);
2) CN-TRANS: Administered a placebo and transported (n=16 subjects);
3) CN-STAY: Administered a placebo and not transported (n=16 subjects).

Calf Sourcing and Transportation: Calves will be sourced from a calf ranch 6-8 hours away from the KSU-CVM. Calves in the IH-TRANS and CN-TRANS groups will be transported as a single group in a truck and trailer. Calves in the CN-STAY group will remain at the source farm.

### B. Test Articles:

Industrial hemp: IH will be administered via gel capsules to target a cannabidiolic acid (CBDA) loading dose of 10 mg/kg and a second maintenance dose of 5.0 mg/kg;
Placebo: Alfalfa meal will serve as a placebo. Alfalfa meal will be placed into gel capsules to an equal volume of IH.

It is estimated each calf will receive between 1-2 boluses. Boluses will be administered via balling gun with the animal restrained either in their individual pens/hutches or chute with head gate.

### Outcome Variables

Outcome parameters to be collected include: cannabinoid concentrations, complete blood counts (CBC), serum biochemistry, plasma cortisol, substance P, prostaglandin E2 Metabolites, serum amyloid A (SAA) and ocular infrared thermography at predetermined time points.

Proposed time points are just prior to transport (TO); at arrival to KSU (˜8 h), and 168 h (7 d) post-dosing/loading. For calves in the CN-STAY group, study team members will remain at the source farm to collect samples when the TRANS groups arrive at KSU. Calves will be administered their respective test article (industrial hemp or alfalfa meal) 12 h prior to loading and just prior to loading onto the trailer. At each time point, cattle will be moved into a chute and head restrained in a head catch. Blood will be taken by restraining the head with rope halter or using neck restraints on the chute.

### (1) Blood Sampling:

Whole blood will be obtained prior to IH feeding and transportation (TO), and at arrival to KSU (˜8 h), 168 hours post-loading. Whole blood will be taken from the jugular vein using a 20 mL syringe and 18 ga. 1.5-inch needle. Whole blood will be placed into tube containing EDTA, heparin, or no additive. Outcomes for whole blood include CBC, blood chemistry profile, plasma cannabinoid concentrations, cortisol, substance P, prostaglandin E2 metabolites, and serum amyloid A.

### (a) Complete Blood Count and Biochemistry Profile

Complete blood counts and serum biochemistry profiles will be submitted on each animal to the KSU-VDL Clinical Pathology lab. Whole blood and serum will be analyzed using AAVDL methods in the laboratory.

### (b) Plasma Cannabinoid Concentrations

Plasma cannabinoid concentrations will be analyzed using high pressure liquid chromatography coupled with mass spectroscopy (HPLC-MS) by the KSU Analytical Chemistry Lab.5 The lower level of quantification for 15 cannabinoid compounds (11 cannabinoids and 4 metabolites) 2.5 ng/mL.

### (c) Cortisol

Plasma cortisol concentrations will be determined in duplicate via radioimmunoassay (Corti-Cote Cortisol RIA kit with ANS. Part no. 06B-256440 ICN; MP Biomedical, Santa Ana, CA, USA). The assay has a detection range of 0.64 to 150 ng/ml. Area under the effect curve (AUEC) will be calculated using the linear trapezoidal method.

### (d) Substance P (SubP)

Determination of substance P levels will be determined by radioimmunoassay (RIA) using a validated non-extracted plasma method.7 The method is a double antibody assay using I-125 linked substance P. The assay has a detection range of 4.7 to 600 μg/ml. Samples will be assayed in duplicate with the reported concentration equaling the average SubP concentration between duplicates.

### (e) Prostaglandin E₂ Metabolites (PGEM)

Prostaglandin E₂ metabolites will be determined using a commercially available ELISA kit. The assay has a test range of 0.39 to 50 μg/mL. Samples will be determined in duplicate with the reported concentration equaling the average PGEM concentration between duplicates.

### (f) Serum Amyloid A (SAA)

Serum amyloid A will be determined using a commercially available ELISA kit. The assay has a test range of 9.4 to 150 μg/mL. Samples will be determined in duplicate with the reported concentration equaling the average SAA concentration between duplicates.

### (2) Ocular Infrared Thermography (IRT):

Temperatures of the medial canthus of the eye will be taken via infrared thermography using methods described by our lab. Images will be obtained at a distance of approximately 0.5 m from the eye. Image file number will be recorded and used for later analysis using computer software. Infrared images will be analyzed using research grade computer software (SmartView 4.3, Fluke Corp., Everett, WA) to determine maximum, minimum and average temperatures of the eye. Two IRT cameras (Fluke Ti580, Fluke Corp., Everett, WA) are available for use.

### (3) Activity Monitoring:

Activity during transportation will be done in the IN-TRANS and CN-TRANS groups using IceQube accelerometers (IceRobotics Ltd, South Queensferry, Edinburgh, Scotland UK). Accelerometers will be places on the left rear leg per manufacture recommendation at the time of enrollment and remain on until the 7 d time point. Steps, time standing up, time lying down and lying bouts, and motion index data was collected via accelerometers at 15-minute intervals. Raw data was downloaded using a RFID reader and computer software (IceManager 2014, IceRobotics Ltd, South Queensferry, Edinburgh, Scotland UK).

### Study Statistics:

For sample size determination, cortisol was determined to be the variable of interest based on data generated by our group. Based on cortisol data from our lab group from Holstein cattle either administered IH for a target CBDA dose of 5.5 mg/kg or no hemp control a true difference of 4.06 ng/mL was found with a standard deviation of 1.99 ng/ml. To achieve a power of 0.80 and alpha of 0.05, ten calves per treatment group are needed.

All statistical analysis will be performed using statistical software (JMP Pro 15.1, SAS Institute, Cary, NC). Outcome variables will be analyzed using a mixed linear model with the calf as the experimental unit. Treatment, time, and treatment by time interaction will be analyzed. A post hoc test will be conducted using the Tukey-Kramer adjustment for significant outcomes. Statistical significance will be set at p<0.05.

1. A method of improving performance, minimizing negative effects of stressful stimuli, or improving stress response, behavior, immune function, morbidity, performance, and/or carcass characteristics of livestock, said method comprising administering an effective amount of cannabinoids to an animal before, during and/or after exposure to a stressful event, wherein said cannabinoids are administered to said animal for a total time period of from 5-14 days.

2.-3. (canceled)

4. The method of claim 1 wherein said cannabinoids are selected from the group consisting of cannabidiolic acid (CBDA), cannabidivarinic acid (CBDVA), cannabidiol (CBD), cannabichromenic acid (CBCA), cannabigerolic acid (CBGA), and tetrahydrocannabinolic acid A (THCA-A), and combinations thereof.

5. The method of claim 1 wherein said cannabinoids are administered as a pre-transport and/or on arrival therapeutic for an animal transported to a feedlot.

6. The method of claim 1 wherein said animal has improved performance after the stressful event.

7. The method of claim 1 wherein said animal has reduced cortisol as compared to a control.

8. The method of claim 1 wherein said animal has reduced PGE₂ as compared to a control.

9. The method of claim 1 wherein said animal has increased resting time as compared to a control.

10. The method of claim 1 wherein said cannabinoid is administered for 5-7 days.

11. The method of claim 1 wherein said animal is a ruminant or pre-ruminant animal.

12. The method of claim 1 wherein said cannabinoid is administered to provide an effective amount of 0.5 mg/kg bodyweight to about 30 mg/kg of bodyweight of the animal.

13. An animal feed or feed additive for use in a method according to claim 1, said animal feed or feed additive comprising an effective amount of said cannabinoids.

14. The animal feed of claim 13, further comprising one or more taste enhancers.

15. The animal feed of claim 1, wherein said feed additive is administered as a top dressing on animal feed.

16. The animal feed of claim 1, comprising industrial hemp comprising said cannabinoids.

17. The animal feed of claim 1, comprising isolated and/or concentrated cannabinoids.
